(19) United States
(12) Patent Application Publication
(10) Pub. No.: US 2002/0173505 A1
(43) Pub. Date: Nov. 21, 2002

(54) Medicament

(76) Inventor: Staffan Skogvall, Lund (SE)

Correspondence Address: SMITH, GAMBRELL & RUSSELL, LLP, ATTORNEYS AT LAW, SUITE 800, 1850 M STREET, N.W., WASHINGTON, DC, 20036, US

(21) Appl. No.: 09/984,329

(22) Filed: Oct. 29, 2001

Related U.S. Application Data
(60) Provisional application No. 60/131,355, filed on Apr. 28, 1999.
(60) Provisional application No. 60/136,604, filed on May 27, 1999.
(60) Provisional application No. 60/139,632, filed on Jun. 17, 1999.
(60) Provisional application No. 60/139,633, filed on Jun. 17, 1999.

(30) Foreign Application Priority Data
Apr. 28, 1999 (SE) 9901531-5
May 26, 1999 (SE) 9901906-9
Jun. 15, 1999 (SE) 9902251-9
Jun. 15, 1999 (SE) 9902252-7
Apr. 28, 2000 (SE) PCT/SE00/00819

(51) Int. Cl. A61K 31/551; A61K 31/506; A61K 31/519; A61K 31/495

(52) U.S. Cl. 514/220; 514/252.14; 514/255.03; 514/254.09; 514/259.41; 514/411; 514/278; 514/288; 514/419; 514/314

(57) ABSTRACT

A compound having agonist activity to the 5-HT₄ receptor for use as a medicament and the use of said compound in the manufacture of a medicament for use in therapeutic or prophylactic treatment of disorders involving bronchocontraction of a human or animal body is described, as well as methods of treatment, wherein said compounds are administered. Further, a compound having antagonist activity to the 5-HT₂a receptor for use as a medicament and the use of said compound in the manufacture of a medicament for use in therapeutic or prophylactic treatment or disorders involving bronchocontraction of a human or animal body is described, as well as methods of treatment, wherein said compounds are administered. Moreover, a composition comprising a combination of compounds comprising at least one compound with agonist activity to the 5-HT₄ receptor agonist and at least one compound with antagonist activity to the 5-HT₂, receptor antagonist is described, as well as such a composition for use as a medicament, the use of said composition for the manufacture of a medicament for therapeutic or prophylactic treatment of disorders involving broncocontraction of a human or animal body, and methods of treatment, wherein said compositions are administered.

### RELATED ART

The present application claims priority from the following U.S. provisional applications: 60/131,355; 60/136,604; 60/139,632; and 60/13R,633, as well as from the following Swedish applications: 9901531-5; 9901906-9; 9902251-9; and 9902252-7.

### FIELD OF THE INVENTION

The present invention relates to a compound having agonist activity to the 5-HT₄ receptor for use as a medicament and to the use of said compound in the manufacture of a medicament for therapeutic or prophylactic treatment of disorders involving bronchocontraction of a human or animal body, as well as methods of treatment, wherein said compound is administered. The present invention also relates to a compound having antagonist activity to the 5HT2a receptor for use as a medicament and to the use of said compound in the manufacture of a medicament for therapeutic or prophylactic treatment of disorders involving bronchocontraction of a human or animal body, as well as methods of treatment, wherein said compound is administered.

### BACKGROUND OF THE INVENTION

Receptors of the 5-HT (serotonin; 3-(P-aminoethyl)-5-hydroxyindole) type are well known and occur throughout the body, e.g. in the airways, and their relevance has mainly been reported in conjunction with treatment of CNS, muscle and gastric disorders, as disclosed in e.g. WO 98/18458 and U.S. Pat. No. 5,246,935. In such treatments, compounds having agonist activity to a 5-HT₁ type receptor are often used. As examples of other 5-NT receptors, mention can be made of receptors of the 5-HT₂, 5-HT₄, 5-HT₅, 5-HT6 and 5-HT₇ type. For a recent review of 5-HT receptors, see Gerhardt, C. C. van Heerikhuizen, H., Eur. J. Phar., 334, 1-23 (1997), which is incorporated herein by reference.

Receptors of the 5-HT₂ type are also well known, e.g. through U.S. Pat. Nos. 5,869,497, 5,705,519 and 5 246 935. The relevance of receptors of the 5-HT₂ type has been reported in conjunction with e.g. CNS and neuronal disorders. Such disorders are often treated with compounds having antagonist activity to a receptor of the ⁵-HT₂ₐ, 5-HT_{2B} or 5-HT₂c type. Examples of such compounds are ritanserin and naftidroturyl. A review of typical agonists and antagonists of various 5-HT receptors is disclosed in R. A. Glennon, Neuroscience and Biobehavioral Reviews, 14, 35-47 (1990), the whole content of which is incorporated herein by reference.

SU 1 701 320 A1 discloses the use of serotonin for treatment of acute asthma attacks. This reference does not suggest any receptor mechanism for serotonin, which is a compound with both a contracting and a relaxing effect on the airways, as is further discussed herein below.

In the RBI Handbook or Receptor Classification and Signal Transduction, 3^{rd} Edition, 1998, RBI, One Strathmore Road, Natick, MA 01760-2447, USA, Editor: Keith J. Watling are compounds having agonist or antagonist activity to various receptors disclosed.

### DISCLOSURE OF THE INVENTION

The present invention is based on the novel finding that certain 5-HT receptors are of utmost importance in regulating bronchocontraction. In summary, it is disclosed herein that compounds having agonist activity to the 5-HT₄ receptor bring about a bronchorelaxing action upon administration thereof, and are therefore suitable as agents for treatment of bronchocontraction disorders. It is also disclosed herein that compounds having antagonist activity to the 5-HT₂, especially 5-HT₂ₐ, receptor, are suitable agents in the treatment of bronchocontraction disorders. Methods for treatment of bronchocontraction disorders are also disclosed.

As used herein, the expression bronchocontraction disorder refers to an abnormal increase of the force development of the smooth muscle, resulting in a reduced diameter in same or all of the airways of the lungs and/or the extrapulmonary airways. Said expression also refers to reduction of airflow caused by swelling, oedema, plasma extravasation or mucous secretion caused by e.g. asthma or any other disorder related thereto.

Accordingly, the present invention relates, in one of its aspects, to a compound having agonist activity to the 5-HT₄ receptor for use as a medicament. In another aspect it relates to use of said compound in the manufacture of a medicament for therapeutic or prophylactic treatment of a human or animal body, wherein the medicament is intended for treatment of disorders involving bronchocontraction, such as asthma.

In a preferred embodiment, the invention relates to the use of a compound having agonist activity to the 5-HT₄ receptor in the manufacture of a medicament for therapeutic or prophylactic treatment of disorders involving bronchocontraction, wherein said agonist has the capacity of reducing the pathological bronchocontraction by at least 30t, preferably at least 60%, and most preferably at least 90%.

The present invention also relates, in another aspect, to a compound having antagonist activity to the ⁵-KT2a receptor for use as a medicament. In another aspect it relates to use of said compound in the manufacture of a medicament for therapeutic or prophylactic treatment of a human or animal body, wherein the medicament is intended for treatment of disorders involving bronchocontraction, such as asthma,

In a preferred embodiment, the invention relates to the use of a compound having antagonist activity to a 5-HT₂, receptor in the manufacture of a medicament for therapeutic or prophylactic treatment of disorders involving bronchocontraction, wherein said antagonist has the capacity of reducing the pathological bronchocontraction by at least 30%, preferably at least 60%, and most preferably at least 90%.

Said bronchocontraction may also occur in conjunction with such disorders as e.g. emphysema, chronic bronchitis, chronic obstructive pulmonary disease, depression, anorectic or bulimic eating disorders, anxiety or various psychotic conditions, including schizophrenia.

The present invention also relates to the use of a compound having antagonist activity to a 5-HT₂ₐ receptor in combination with a compound having agonist activity to the 5-HT₄ receptor in the manufacture of a medicament for therapeutic or prophylactic treatment of disorders involving bronchocontraction. In a preferred embodiment said compound having agonist activity is serotonin or a derivative thereof having agonist activity to the 5-HT₄ receptor. This combination of the 5-HT2a receptor antagonist and the agonist increases the serotonin transmission in the body, particularly in the presence of a serotonin uptake inhibitor (SRI) - Further, the compounds having agonist activity to the 5-HT₄ receptor to be used according to the present invention are also useful in the present combination embodiment. In particular, said medicament is intended for treatment of asthma and disorders related thereto.

According to the present invention several known substances are, surprisingly, able to stimulate the 5-HT₄ receptor, without activating the contracting 5-HT₂ₐ receptor, thereby generating a relaxing effect on the bronchocontraction. Such agonist compounds are selected from the group comprising the substances SC 53116, ML 10302, RS 67506 and BIMU 8, which are defined below, as well as the more unspecific 5-carboxamidotryptamine, and derivatives and pharmaceutically acceptable salts thereof having the same or essentially the same relaxation effect.

The invention also relates to the use of one or more of the above-mentioned agonist compounds: SC 53116, i.e. 4-amino-5-chloro-N-[[lS, 7aS)-hexahydro-1H-pyrrolizin-l-yl]methyll-2-methoxy-benzamide, having the structural formula:

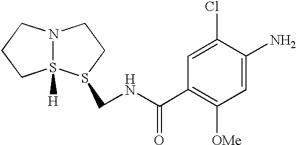

4-amino-S-chloro-2-methoxybenzoic acid esters, e.g. RS 57639, SR 59768, and ML 103.O2, i e 4-anino-5-chloro-2-methoxy-benzoic acid-2-(l-piperidinyl)ethylester, having the structural formula:

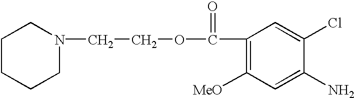

RS 6750G, i.e. M-[2-[4-[3-(4-amino-5-chloro-2-methoxyphenyl) -3-oxopropyl] -1-piperidinyl] ethyl] -methanesulfonamide monohydrochloride, having the structural formula:

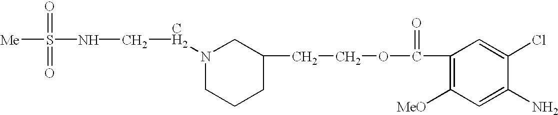

BIMU 8, i.e. 2,3-dihydro-N-[(3-endo)-8-methyl-8-azabicyclo[3.2.1]oct-3-yl)-3-(1-methylethyl) -2-oxo-1H-benzimidazole-1-carboxamide monohydrochloride, having the structural formula:

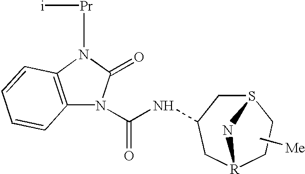

5-carboxamidotryptamine, having the structural formula:

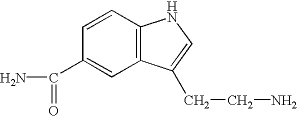

BIMU 1, ERL 24924, Cisapride, DAU 6236, 5-hydroxy-N,N-di-metyltryptamin, ML-1035, ML10302, 5-metoxytryptamin, Metoclopramide, Mosapride, a-OH-DPAT (8-hydroxy-2-dipropylaminotetralin), Prucalopride, R 076186, R 093877, Renzapride, RS 17017, RS 56532, RS 67333, RS 67532, SB 204070, SB 205149, SC-49518, SK-951, SDZ 216-454, 4-amino-5-chloro-2-methoxy-N-((2S, 4S) -l-ethyl-2-hydroxymethyl-4-pyrrolidinyl)bensamid, (e.g. TKS159), 2-piperazinylbenzo-thiazole derivatives (e.g. VB20B7), YM-47813, YM-53389, YM-09151, Zacopride, Zelmac, arylcarbamate derivatives of 1-piperidineethanol, thiophene carboxamide derivatives 3 (a-j), 5.azabicyclo(x.y.z) derivatives, 2-piperazinyl-benzoxazole derivatives, clebopride, and Sandoz compound 1b,

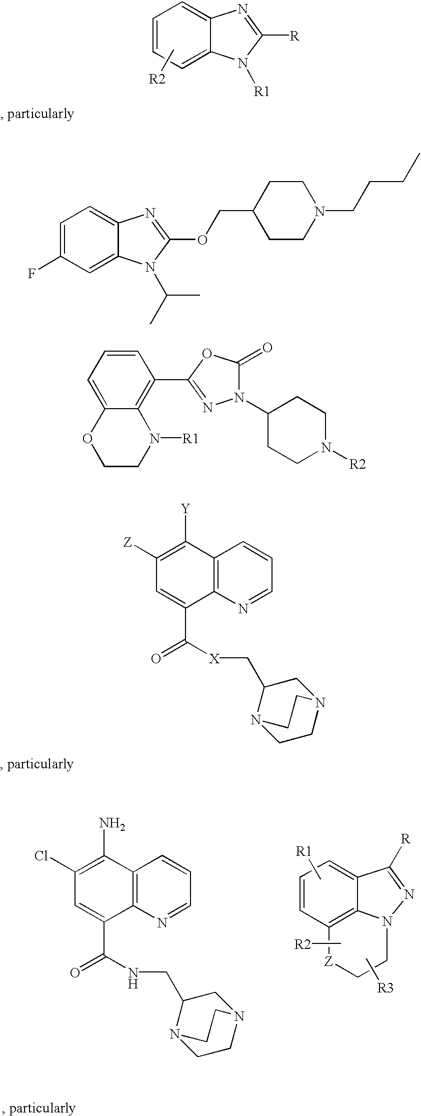

and derivatives and pharmaceutically acceptable salts thereof having essentially the same relaxing effect, in the manufacture of a medicament for therapeutic or prophylactic treatment of disorders involving bronchocontraction, wherein said agonist has the capacity of reducing the bronchocontraction by at least 30%, preferably at least 60%, most preferably at least 90%.

According to the present invention several known antagonist compounds are, surprisingly, able to influence the 5-HT2a receptor, thereby generating a contraction reducing effect, i.e. a relaxation effect, and are selected from a group comprising ketanserin, AMI-193 or MDL 100 907, and derivatives and pharmaceutically acceptable salts thereof having the same or essentially the same contraction reducing effect.

Thus, the invention also relates to the use of one or more of the above-mentioned compounds, namely: ketanserin, i.e 7-azido-3-[2-[4-(4-fluorobenzoyl)-1-piperidinyl]ethyl]-6-iodo-2,4(1H, 3H)-Quinazolinedione, having the structural formula:

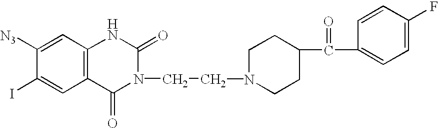

AMI-193, i.e. S-[3-(4-fluorophenoxy)propyl]-l-phenyl-1,3,B-triazaspiro[4,5]decan-4-one, having the structural formula:

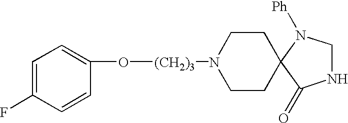

ALEPH-2, Amperozide, amesergide, aryloxyalkylimidazolines, 1-aryl-4-propylpiperazines, BIMT 17, 1-3-(4-(3-chlorophenyl)-1-piperazinyl]propyl-6-fluoroindolin-2(1 H)-one, CGS 18102A, Clonidine, Cyproheptadine, Deramciclane, Desmethyl-WAY 100635, dotarizine, DV 7028, Elymoclavine, Fananeerin, 8- [3- (4-fluorobenzoyl) propyl] -1-rnethyl-1,3,8-triazaspiro[4,5]decan-4-one, FG5893 hydrochloride, FG5974, F05983, hexahydrocarbazoles, (3H)WAY 100635, ICI 169,369, 8-[3-(4-iQdobenzoyl)propyl]-1-methyl-1,3,8-triazaspiro (4,5]decan-4-one, LEK-8804, LSD, LU 111995, (8,9S)-LY-53,857, (R,S)-LY-53,857, (S,R)-LY-53,857, (R,R)-LY-53,857, LY-53,857 free base, LY 215840, MDL-11,939, MDL 28133A, MDL 100,151, MDL 100,907, mesulergine, Metergoline, 1-3-[4-(2-methoxy-phenyl)-1-piperazinyl]propyl indolin-2(1H)-one, methysergide, Mianserin, NE-100, Nefazodone, N-ethoxycarbonyl-2-ethoxy-1,2-dihydroquinoline, NRA0045, Olanzapine, Ondansetron, 1-(2-pyritnidinyl)piperazine derivatives, Pizotifen, raclopride, Roxindole, Risperidone, Ritanserin, RP62203, sarpogrelate and its active metabolite (M-1), serotonin reuptake inhibitors like fluoxetine, YM 992, medifoxamine, cericlamine, imipramine, iprindole, BIMT 17, citalopram, paroxetine, sertraline, fluvoxamine spiro indoles N-substituted with a 3-(dimethylamino)-propyl chain, Spiperone, SR 46349B, WAY 100635, WY-50,324, and derivatives and pharmaceutically acceptable salts thereof having essentially the same contraction reducing effect, in the manufacture of a medicament for therapeutic or prophylactic treatment of disorders involving bronchocontraction, wherein said antagonist has the capacity of reducing the pathological bronchocontraction by at least 30%, preferably at least 60%, and most preferably at least 90%.

Ketanserin is excluded from the embodiment concerning the 5-HT₂ receptor antagonist compound for use as a medicament.

The present invention also relates to a method for treatment of disorders involving bronchocontraction, wherein said method comprises administering to a human or animal patient a therapeutically effective amount of the compound according to the present invention having agonist activity to the 5-ET₄ receptor. Preferably, said method relates to the treatment of asthma and disorders related thereto.

The present invention also relates to a method for treatment of disorders involving bronchocontraction, wherein said method comprises administering to a human or animal patient a therapeutically effective amount of a compound according to the present invention having antagonist activity to a 5-HT₂a receptor. Preferably, said method relates to treatment of asthma and disorders related thereto.

Further, the present invention relates to a method for treatment of disorders involving bronchocontraction, wherein the above-mentioned combination of agonist(s) and antagonist(s) is administered.

The expression “has the capacity of reducing the pathological bronchocontraction by at least . . . %” used throughout the present patent application means that the compound in question reduces the contraction in the airways caused (1) either by the underlying disease (asthma etc) or (2) by the administration of 5-HT or other substances with 5-HT₂ₐ-activating properties. The level of contraction in the airways can, for instance, be determined by spirometric measurements of the Forced Expiratory Volume (FEV1), compared to the normal value for healthy people. Alternatively, the expiratory capacity for a patient can be compared to his own FEV1 during periods of relatively little obstructive problems.

As appears from FIG. 1, the contractile component often manifests itself as a reduction or a complete elimination of the 5-HT induced relaxation, rather than in an increase of force from the control (pre-exposure) level. In the case of “specific” agonists to the 5-HT₄ receptor, this sustained relaxing effect is achieved because the contractile 5-HT2a receptor is not affected; only the relaxing 5-HT₄ receptor is activated. In the case of antagonists to the 5-HT₂a receptor, this effect is achieved due to direct blocking of the 5-HT₂-, receptor, whereby the unspecific agonists to the 5-HT₄ receptor, such as 5-HT, can act without also causing contraction by the 5-HT₂, receptor.

It should be noted that the medicament prepared according to present invention in each embodiment may optionally include two or more of the above outlined compounds, Furtherr in the embodiment when the compound having 5-HT₂, antagonist activity is administered, optionally together with complementary serotonin or derivatives thereof, a serotonin uptake inhibitor can be added with a view to amplifying the relaxing effect.

The typical daily dose of the medicament prepared according to the invention varies within a wide range and will depend on various factors such as the individual requirement of each patient and the route of administration.

Said medicament may be prepared as a composition adapted either for administration via the respiratory tract or for oral, intravenous, topical, intraperitoneal or subcutaneous administration, in association with one or more pharmaceutically acceptable carriers, diluents or adjuvants that are well known in the art.

Moreover, said medicament is preferably administered via the respiratory tract in the form of e.g. an aerosol or an air-suspended fine powder. However, in some cases a useful alternative to administration via the respiratory tract may be oral, topical, parenteral, subcutaneous, transdermal or rectal administration, wherein e.g. tablets, capsules, powders, microparticles, granules, syr- ups, suspensions, solutions, transdermal patches or sup- positories are utilized.

### BRIEF DESCRIPTION OF THE DRAWINGS

FIG. 1 depicts the effects of 5-HT and selective 5-HT₄ agonists on the spontaneous tone in human in vitro preparations. Note that 5-HT only gives a transient relaxation, while selective 5-HT₄ agonists give a strong sustained relaxing effect.

### DETAILED DESCRIPTION

The subject-matter of the present invention was inter alia deduced from animal experiments, where a specific behavior of the airway smooth muscle called “spontaneous tone” was examined. The spontaneous tone, which involves a spontaneous continuous contraction in the airway smooth muscle, was studied due to a suspicion that detective regulation of the spontaneous tone could be an important cause of the bronchoconstriction observed in asthmatic patients.

The examinations of the spontaneous tone were performed in accordance with the methods disclosed in the thesis “Regulation of spontaneous tone in guinea pig trachea” by S.Skogvall, Department of Physiological Sci- ences, Lund University, 1999, which is incorporated herein by reference. As evidenced by these examinations, the airways normally display a highly regular type of oscillating tone if exposed to physiological conditions, and the oscillating tone can be reversibly affected by administration of various substances- When the epithelium is removed, the preparations instead display a strong, smooth type of tone. In short, the animal experiments in said thesis showed that the spontaneous tone to a large degree is controlled by powerful regulating factors released from neuroepithelial endocrine (NEE) cells.

Later experiments, not included in the thesis, have revealed that one of the regulating factors is serotonin, also called 5-HT, which exerts agonist action on the receptors ceptors 5-HT₄, 5HT₅, 5-HT₆, 5-HT₇ as well as on 5-HT₂ receptors.

Additional experiments have shown that when 1 μM serotonin was added to denuded airway smooth muscle preparations from the guinea-pig displaying a strong, smooth spontaneous tone, the average force level was increased significantly, i.e. a contraction was observed. A contractile effect of serotonin on airway smooth muscle has been reported in e.g. SkQgvall, S., Korsgren, M-, Grampp, W., J. Appl. Phys., 86:789-798, 1999. However, when 10 μM of serotonin was added, the spontaneous tone was significantly suppressed to a level of about half the force observed in control (drug-free) conditions. The spontaneous tone returned to approximately its normal level when the preparations were again exposed to control conditions. Thus, it has now surprisingly been shown that serotonin brings about contraction of the airways at low concentrations and relaxation at high concentrations, consequently having a dual effect on the airways.

Furthermore, it has been shown that when the contracting 5-HT2, receptor was blocked with ketanserin, the S-ST, i.e. serotonin, induced almost no contraction, but instead only a significant relaxation. Similar experiments have also been performed on human in vitro preparations, from patients undergoing lobecotomy or pulmectomy due to lung cancer. It was found that in this tissue, 5-HT was even more potent in relaxing the airway smooth muscle than in guinea pig. In human tissue, already 1 μM 5-HT induces a significant relaxation of the spontaneous tone.

Human airways are generally considered to display only a weak contraction when exposed to S-HT. Nevertheless, examinations on spontaneous tone on human in vitro preparations have shown that 5-HT indeed has a contractile component also in this tissue. However, this contraction takes a longer time to develop than in guinea pig and the contractile effect is seen as a termination of the relaxation, rather than an increase of tone from the baseline. In guinea pig trachea, the contraction reaches a maximum after approximately 10 min, and this is followed by a considerable reduction of tone. However, human preparations instead induce a maximum relaxing effect after 5-10 min, which disappears gradually during the following 30-45 min (see FIG. 1). The transient nature of the 5-ET relaxation is most likely caused by a simultaneous activation of the fast, relaxing 5-HT₄ receptor, and a slower activation of the contracting SHT₂a receptor. This is clear, because activation of the relaxing 5-HT₄ receptor by a Substance that lacks 5-HT₂, receptor activating properties (such as 5-carboxiamidotryptamine or SC 53116), results in a relaxation that is persistent and not transient (see FIG. 1).

It has previously been suggested that b-HT or B-HT analogues may be useful in the treatment of bronchoobstructive diseases. In SU 1 701 320 it is suggested that the 5-Hr₁ i.e. serotonin, may be of use as an addition to standard beta2 receptor stimulation. However, from our experiments it seems clear that S-HT is not effective or useful as the only treatment for e.g. asthmatic disorders, because of the transient relaxing effect by S-HrT (see FIG. 1). If instead, as we propose herein, a 5-HT analogue that lacks the 5-HT2a activating properties is given, the relaxing effect is persistent, and not transient.

In summary, it has now been discovered that agonist action on the 5-HT₄ receptor results in a relaxing effect, whereas agonist action on 5-HT2a receptors results in a contractile effect. In conclusion, the dual effect of serotonin is most likely a result of its agonist action on the relaxing 5-UT₄ receptor as well as on the contracting 5-HT₂7 receptor.

It was also deduced from these experiments that compounds having agonist activity to the 5-HT₄ receptor, while having only low or no agonist activity to a 5-HT₂a receptor, therefore are useful as agents for treatment of bronchocontraction disorders.

Thus, the present invention relates to the use of compounds having agonist activity to the 5-HT₄ receptor in the manufacture of a medicament intended for treatment of bronchocontraction disorders, whereby said compounds have the strong bronchorelaxing effect of serotonin but have substantially no contractile effect. As mentioned above, the compounds used according to the present invention have only low or no agonist activity to 5-HT₂a receptors.

In the above mentioned experiments it has also been shown that compounds having antagonist activity to a 5-HT2. receptor are useful as agents for treatment of bronchocontraction disorders, since they are capable of blocking the contractile effect of a compound having agonist activity to a 5-HT₂, receptor. The compounds according to the present invention having antagonist activity to the 5-HT₂, receptor may even be administered together with serotonin in the form of a complement so the serotonin content already present in the body with a view to obtaining an amplified contracting effect, or with any other substance having agonist activity to the 5-HT2a receptor; or with a serotonin uptake inhibitor Said administration can be simultaneous or sequential, and a powerful relaxing effect on the bronchi can be achieved in this manner. Thus, the present invention also relates to the combined use of a compound having antagonist activity to a 5-HT2,-receptor and a compound having agonist activity to the 5-HT₄ receptor, in the manufacture of a medicament for therapeutic or prophylactic treatment of disorders involving bronchocontraction.

1. Use of one or more compounds having antagonist activity to a 5-HTI. receptor, and derivatives and pharmaceutically acceptable salts thereof having antagonist activity to the 5-HT₂. receptor in the manufacture of a medicament for therapeutic or prophylactic treatment of disorders involving human bronchocontraction chosen from the group consisting of asthma and disorders related thereto, emphysema, chronic bronchitis, and chronic obstructive pulmonary disease.

2. Use according to claim 1, wherein said compounds have the capacity of reducing pathological bronchocontraction by at least 30%, preferably at least 60%, and most preferably at least 90%, and wherein said compounds are chosen from the group comprising AMI-193 and MDL 100,907, ALEPH-2, Amperozide, amesergide, aryloxyalkylimidazolines, 1-aryl-4-propylpiperazines, BIMT 17, 1-3-[4-(3-chlorophenyl)-1-piperazinyl]propyl-S-fluoro-indolin-2(l H)-one, CGS 18102A, Cyproheptadine, Deramciclane, Desmethyl-WAY 100635, dotarizine, DV 7028, Elymo-clavine, Fananserin, 8-[3-(4-fluorobenzoyl)propyll-1- methyl-1,3,8-triazaspiro[4,51decan-4-one, FG5893 hydro-chloride, FG5974, FG5983, hexahydrocarbazoles, (3H)WAY 100635, ICI 169,369, a-[3-(4-iodobenzoyl)propyl]-1-methyl-1,3,8-triazaspiro[4,5]decan-4-one, LEK-8804, LSD, LU 111995, (S,S)-LY-53,85?, (?,S)-LY-53,8s7, (S,R)-LY-53,857, (R,R)-LY-53,857, LY-53,8-57 free base, LJY 215840, MDL-11,939, MDL 28133A, MDL 100,151, MDL 100,907, mesulergine, Metergoline, 1-3-[4-(2-methoxyphenyl)-1-piperazinyllpropyl indolin-2 (1H) -one, methysergide, Mianserin, NE-100, Nefazodone, N-ethoxycarbonyl-2-ethoxy-i,2-dihydroquinoline, NRA0045, olanzapine, Ondansetron, 1-(2-pyrimidinyl) piperazine derivatives, Pizotifen, raclopride, Roxindole, Risperidone, Ritanserin, RP62203, sarpogrelate and its active metabolite (M-1), serotonin reuptake inhibitors like fluoxetine, YM 992, meditoxamine, cericlamine, imipramine, iprindole, BIMT 17, citalopram, paroxetine, sertraline, fluvoxamine Spiro indoles N-substituted with a 3-(dimethylamino)propyl chain, Spiperone, SR 46349B, WAY 100635, and WY-50,324, and derivatives and pharmaceutically acceptable salts thereof.

3. Use according to claim 2, wherein said compound is AMI-193, MDL 11,939, WAY 100635r Spiperone, Pizotifen, Risperidone, Ritanserin, Fluoxetin, Fluvoxamin, or FG 5963.

4. Use according to any one of claims 1-3, wherein said disorder involving bronchocontraction is asthma and disorders related thereto.

5. A method for treatment of disorders involving bronchocontraction, wherein said method comprises administering to a human or animal patient, suffering from asthma and disorders related thereto, emphysema, chronic bronchitis, and chronic obstructive pulmonary disease, a therapeutically effective amount of a compound according to any one of claims 1-3.

6. Use of one or more compounds having agonist activity to a 5-HT₄ receptor in the manufacture of a medicament for therapeutic or prophylactic treatment of disorders involving human bronchocontraction, chosen from the group consisting of asthma and disorders related thereto, emphysema, chronic bronchitis, and chronic obstructive pulmonary disease, wherein said compounds have the capacity of reducing pathological bronchocontraction by at least 30%, preferably at least 60%, and most preferably at least 90%, and wherein said compounds are chosen from the group comprising 5-carboxamidotryptamine, BIMU 1, BIMU 8, BRL 24924, Cisapride, DAU 6236, 5-hydroxy-N,N-dimetyltryptamin, ML-1035, ML 10302, 5-metoxytryptamin, Metoclopramide, Mosapride, 8-OH-DPAT (8-hydroxy-2-dipropylaminotetralin), Prucalopride, R 076186, R 093877, Renzapride, RS 17017, RS 56532, RS 57639, RS 67333, RS 67506, RS 67532, SB 204070, SB 205149, SC-53116, SC-49518, SK-951, SDZ 216-454, SR59768, TKS159, VB20B7, YM-47813, YM-53389, YM-09151, Zacopride, Zelmac, arylcarbamate derivatives of 1-piperidineethanol, 2-piperazinylbenzoxazole derivatives, clebopride, and 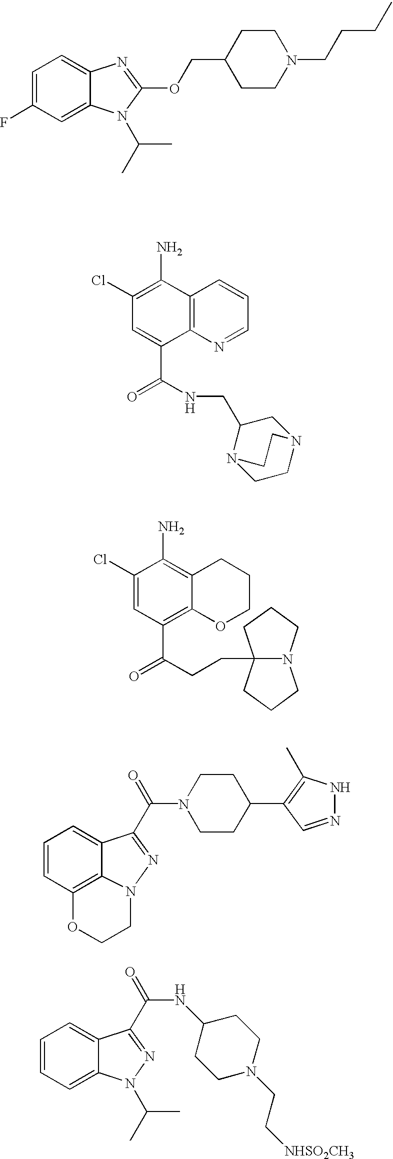
and derivatives and pharmaceutically acceptable salts thereof,

7. Use according to claim 6, wherein said compound is VB20B7, RS 67333, BIMU 1, nIMU 8, 5-methoxytryptamine, Zacopride, RS 56532, Mosapride, BRL 24924, or SC-53116.

8. Use according to any one of claims 6 and 7, wherein said disorder involving bronchocontraction is asthma and disorders related thereto.

9. A method for treatment of disorders involving bronchocontraction, wherein said method comprises administering to a human or animal patient, suffering from asthma and disorders related thereto, emphysema, chronic bronchitis, and chronic obstructive pulmonary disease, a therapeutically effective amount of a compound according to any one of claims 6 and 7.

10. Use of a composition comprising a combination of at least one compound with agonist activity to the 5-HT-₄ receptor, and at least one compound with antagonist activity to the 5-HT2a receptor for the manufacture of a medicament for therapeutic or prophylactic treatment of disorders involving bronchocontraction, chosen from the group consisting of asthma and disorders related thereto, emphysema, chronic bronchitis, and chronic obstructive pulmonary disease, preferably asthma and disorders related thereto.

11. Use according to claim 10, wherein said composition has the capacity of reducing pathological bronchocontraction by at least 30%, preferably at least 60%, and most preferably at least 90%, and wherein said combination is chosen from the following groups of a) 5-HT₄ receptor agonists, and b) 5-HT₂. receptor antagonists, or derivatives or pharmaceutically acceptable salts thereof: a) 5-HT₄ receptor agonists: 5-carboxamidotryptamine, lIMU 1, BIMU 8, BRL 24924, Cisapride, DAU 6236, 5-hydroxy-N,N-dimetyltryptamin, ML-1035, ML 10302, 5-metoxytryptamin, Metoclopramide, Mosapride, 8-OH-DPAT (8-hydroxy-2-dipropylaminotetralin), Prucalopride, R 076186, R 093877, Renzapride, RS 17017, RS 56532, RS 57639, RS 67333, RS 6750G, RS 67532, SB 204070, SE 205149, SC-53116, SC-49518, SK-951, SDZ 216-454, SR59768, TKS159, VB20B7, YM-4781,3, YM-53389, S YM-09151, Zacopride, Zelmac, arylcarbamate derivatives of I-piperidineethanol, 2-piperazinylbenzoxazole derivatives, clebopride, and 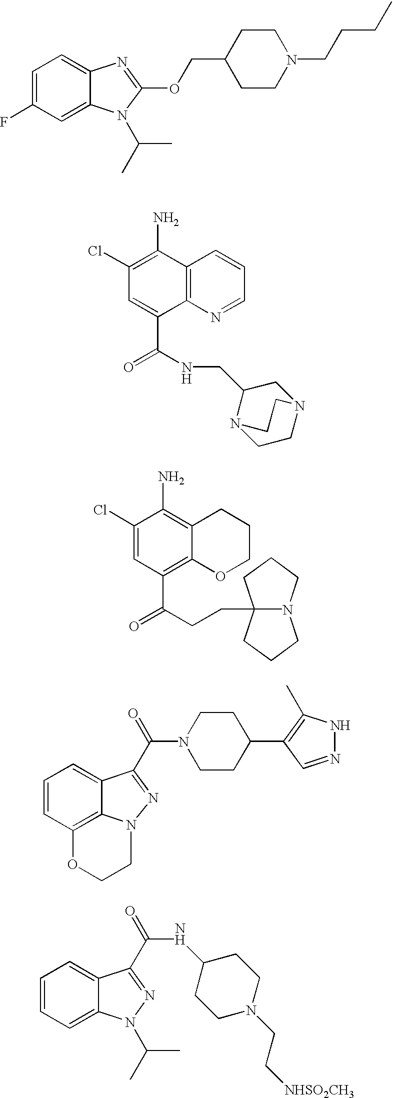
and serotonin (5-HT) and derivatives and pharmaceutically acceptable salts thereof.
b) S-HT₂a receptor antagonists: AMI-193 and MDL 100,907, ALEPH-2, Amperozide, amesergide, aryloxyalkylimidazolines, 1-aryl-4-propylpiperazines, BIMT 17, 1-3-[4-(3-chlorophenyl)-1-piperazinyl ]propyl-8-fluoroindolin-2(1 H)-one, CGS 18102A, Cyproheptadine, Deramciclane, Desmethyl-WAY 100635, dotarizine, DV 7028, 8lymoclavine, Fananserin, 8-[3- (4-fluorobenzoyl)propyll-1-methyl-1,3,8-triazaspiro[4,S]decan-4-one, FG5893 hydrochloride, PG5974, F05983, hexahydrocarbazoles, (3H)WAY 100635, ICI 169,369, 8-[3-(4-iodobenzoyl) propyll-1-methyl-1,3,8-triazaspiro[4,5]decan4-one, Ketanserin, LEK-8804, LSD, LU 111995, (S,S)-LY-53,857, (R,S)-LY-53,857, (S,R)-LY-53,857, (R,R)-LY-53,857, LY-53,857 free base, LY 215840, MDL-11,939, MDL 28133A, MDL 100,151, MDL 100,907, mesulergine, Metergoline, 1-3-14- (2-methoxyphenyl)-1-piperazinyl]-propyl indolin-2(1H)-one, methysergide, Mianserin, NE100, Nefazodone, N-ethoxycarbonyl-2-ethoxy-1,2-diydroquinoline, NRA0045, Olanzapine, Ondansetron, 1-(2-pyrimidinyl) piperazine derivatives, Pizotifen, raclopride, Roxindole, Risperidone, Ritanserin, RP62203, sarpogrelate and its active metabolite (M-1), serotonin reuptake inhibitors like fluoxetine, YM 992, medifoxamine, cericlamine, imipramine, iprindole, BIMT 17, citalopram, paroxetine, sertraline, fluvoxamine spiro indoles N-substituted with a 3-(dimethylamino)-propyl chain, Spiperone, SR 46349B, WAY 100635, and WY-50,324, and derivatives and pharmaceutically acceptable salts thereof.

12. Use according to claim 11, wherein the composition comprises the following combinations of a 5-HT,₄ receptor agonist and a 5-HT₂, receptor antagonist: VB20B7 and AMI-193, VB20B7 and MDL 11939, RS67333 and AMI-193, RS67333 and MDL 11939, VB20B7 and WAY 100635, RS67333 and WAY 100635, Zacopride and AMI-193, Zacopride and MDL 11939, RS56532 and AMI-193, RS56532 and MDL 11939, VB20B7 and Fluvoxamin, RS67333 and FluvoxaTrnin.

13. A method for treatment of disorders involving bronchocontraction chosen from the group consisting of asthma and disorders related thereto, emphysema, chronic bronchitis, and chronic obstructive pulmonary disease, wherein said method comprises administering to a human or animal patient a therapeutically effective amount of a, composition according to any one of claims 10-12.

14. A method for treatment of disorders involving bronchocontraction chosen from the group consisting of asthma and disorders related thereto, emphysema, chronic bronchitis, and chronic obstructive pulmonary disease, wherein said method comprises administering to a human or animal patient a therapeutically effective amount of a 5-HT₄ receptor agonist according to any one of claims 6 and 7 and a 5-HT₂a receptor antagonist according to any one of claims 1-3, either simultaneously or sequentially.
